(19) United States
(12) Patent Application Publication
(10) Pub. No.: US 2006/0199852 A1
(43) Pub. Date: Sep. 7, 2006

(54) Process for the preparation of substituted benzoxazole compounds

(75) Inventor: Silvio Iera, Montreal (CA)

Correspondence Address: COZEN O' CONNOR, P. C., 1900 MARKET STREET, PHILADELPHIA, PA, 19103-3508, US

(73) Assignee: WYETH, Madison, NJ (US)

(21) Appl. No.: 11/368,719

(22) Filed: Mar. 6, 2006

Related U.S. Application Data
(60) Provisional application No. 60/659,138, filed on Mar. 7, 2005.

(51) Int. Cl. C07D 263/60; A61K 31/423

(52) U.S. Cl. 514/375; 548/217

(57) ABSTRACT

The present invention relates to processes for the preparation of substituted benzoxazole compounds, and in particular 2-(3-fluoro-4-hydroxy-phenyl)-7-vinyl-benzoxazol-5-ol. The processes include the vinylation of a substituted benzoxazole compound having an appropriate substitutable moiety.

This application claims benefit of priority to U.S. provisional patent application Ser. No. 60/659,138 filed on Mar. 7, 2005, which is hereby incorporated in its entirety.

### FIELD OF THE INVENTION

The present invention relates to processes for the preparation of substituted benzoxazole compounds, and in particular 2-(3-fluoro-4-hydroxy-phenyl)-7-vinyl-benzoxazol-5-ol. The processes include the vinylation of a substituted benzoxazole compound having an appropriate substitutable moiety.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p351-361(2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERPβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g. disease frequencies, responses to challenge, etc), it is possible that the explanation involves the difference in estrogen levels between males and females.

U.S. Pat. No. 6,794,403, incorporated herein by reference in its entirety, describes the preparation of substituted benzoxazole ERβ selective ligands having the Formula I, infra. Given the importance of these compounds as potential therapeutics, it can be seen that improved processes for their preparation are of great value. This invention is directed to these, as well as other, important ends.

### SUMMARY OF THE INVENTION

In one aspect, the present invention provides processes for the preparation of compounds of Formula I: 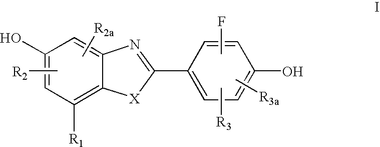
wherein:

R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₅, R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or —SO₂R₅;

or a pharmaceutically acceptable salt thereof;

comprising:

reacting a compound of Formula II: 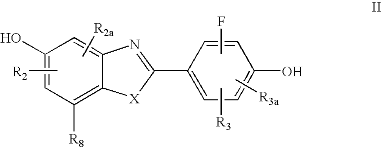
wherein:

R₈ is chloride, bromide, iodide, mesylate, tosylate, triflate, nonaflate or a diazonium salt;

with an alkene having from 2 to about 7 carbon atoms, and which is optionally substituted with hydroxyl, —CN, halogen, trifluoroalkyl, trifluoroalkoxy, —COR₅, —CO₂R₅, —NO₂, —CONR₅R₆, —NR₅R₆ or —NR₅COR₆;

with an alkene;

in the presence of a suitable palladium catalyst and a suitable base, for a time and under the conditions effective to form the compound of Formula I.

In some preferred embodiments, the compound of Formula I has the structure III: 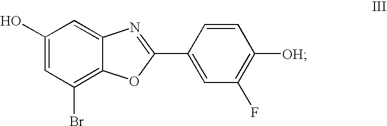
and
the compound of Formula II has the structure IV:

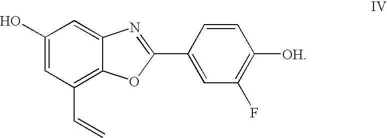

In some embodiments, the alkene is ethylene.

### DETAILED DESCRIPTION OF THE INVENTION

The present invention provides processes for the preparation of a compound of Formula I: 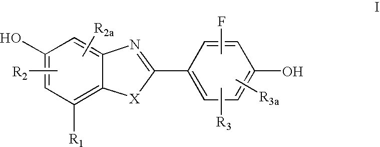
wherein:

R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;

R₅, R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or —SO₂R₅;

or a pharmaceutically acceptable salt thereof;

comprising:

reacting a compound of Formula II: 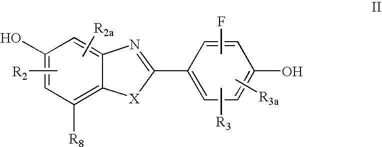
wherein:

R₈ is chloride, bromide, iodide, mesylate, tosylate, triflate, nonaflate or a diazonium salt;

with an alkene having from 2 to about 7 carbon atoms, and which is optionally substituted with hydroxyl, —CN, halogen, trifluoroalkyl, trifluoroalkoxy, —COR₅, —CO₂R₅, —NO₂, CONR₅R₆, NR₅R₆ or NR₅COR₆;

with an alkene;

in the presence of a suitable palladium catalyst and a suitable base, for a time and under the conditions effective to form the compound of Formula I.

In some preferred embodiments of each of the processes described herein, the compound of Formula I has the structure III: 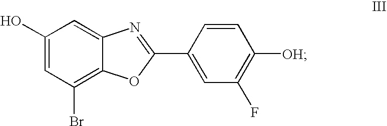
the compound of Formula II has the structure IV: 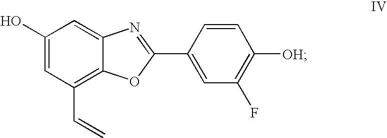
and
the alkene is ethylene.

In accordance with the processes of the invention, the vinylation of the compound of Formula II is accomplished in the presence of a catalyst. Preferably, the catalyst is a palladium catalyst, more preferably comprising a palladium (II) salt and one or more suitable phosphine ligands. One preferred catalyst-ligand combination is palladium diacetate, with tri-o-tolylphosphine. The molar ratio of the ligand to the catalyst is selected such that the desired yield of product is obtained. Typically, the molar ratio of the ligand to the catalyst is from about 1 to about 6; or from about 2 to about 4; or about 3.3.

Generally, the palladium catalyst is present in the reaction mixture in an amount of up to about 5 mole percent, for example up to about 3 mole percent, relative to the compound of Formula II.

A wide variety of bases can be employed in the vinylation reaction. In some embodiments, the base comprises a nitrogen base, for example a trialkyl amine, such as triethylamine. Generally, the base is employed in an amount such that the molar ratio of the base to the compound of Formula II is from about 2 to about 10, for example from about 4 to about 8. In some embodiments, the base comprises triethylene in a molar ratio of base to the compound of formula II of about 4.

Typically, the vinylation reaction is performed in a solvent. While a wide variety of solvents can be employed, polar organic solvents (i.e., solvents including or being composed of at least one polar organic compound) are generally preferred. Some nonlimiting solvents include isopropyl alcohol, dimethylformamide, N,N-dimethylacetamide, 1,2-diethoxyethane, and 1,2-dimethoxyethane. In some preferred embodiments, the solvent includes or is composed of acetonitrile.

Generally, the vinylation reaction is performed at an elevated temperature; i.e., at a temperature greater than room temperature. Typically, a temperature of less than about 100° C. is sufficient to provide acceptable yields of product. Preferably, the vinylation reaction is performed at a temperature of from about 50° C. to about 100° C.; preferably from about 70° C. to about 80° C.

In some embodiments, the alkene is a gas. In such embodiments, it is advantageous to perform the vinylation reaction under a pressure greater than atmospheric pressure. Generally, the pressure is greater than about 30 psi; or greater than about 40 psi, or about 50 psi or greater. In some preferred embodiments, the pressure is about 50 psi.

The vinylation reaction can be performed for any length of time sufficient to provide acceptable yield of product. Generally, a reaction time of up to about 16 hours; or up to about 24 hours, is sufficient.

In some preferred embodiments, the palladium catalyst comprises palladium diacetate and tri-o-tolylphosphine; the molar ratio of tri-o-tolylphosphine to palladium diacetate is from about 2 to about 4; the palladium diacetate is present in an amount of up to about 3 mole percent relative to the compound of Formula II; the base comprises a trialkyl amine; and the molar ratio of the base to the compound of Formula II is from about 4 to about 8. In some especially preferred embodiments, the compound of Formula I has the structure III; and the compound of Formula II has the structure IV.

In some preferred embodiments, the compound of Formula I has the structure III; the compound of Formula II has the structure IV; the palladium catalyst comprises palladium diacetate and tri-o-tolylphosphine; the molar ratio of tri-o-tolylphosphine to palladium diacetate is about 3 to about 4; the palladium diacetate is present in an amount of about 1 mole percent relative to the compound of Formula II; the base comprises triethyl amine; the molar ratio of the base to the compound of Formula II is about 4; the reaction is performed at a temperature of from about 50° C. to about 100° C., preferably from about 70° C. to about 80° C.; the pressure is greater than atmospheric pressure, preferably about 50 psi; and the solvent comprises acetonitrile. In some preferred embodiments, the reaction is performed for up to about 16 hours.

After the vinylation reaction is complete, the product is recovered and purified. In some embodiments, the recovery includes:

a) separating the liquid portion of the reaction mixture from the solid portion;

b) optionally washing the solid portion with an organic solvent and combining the wash with the separated liquid portion;

c) concentrating the liquid portion;

d) extracting the compound of Formula I into an aqueous base solution;

e) acidifying the aqueous base solution; and

f) collecting the compound of Formula I.

The separation of the liquid portion of the reaction mixture from the solid portion can be accomplished by a variety of physical separation techniques. One such technique is by filtration, for example by passing the reaction mixture through a cartridge filtration device.

If desired, the solid portion of the reaction mixture can then be washed one or more times with a solvent to maximize recovery of the liquid portion of the reaction mixture. A variety of wash solvents are suitable, and are easily determined by those of skill in the art. One some preferred embodiments, the wash solvent includes or is composed of 1,2-diethoxyethane.

Typically, the solvent washes are combined with the liquid portion, and the compound of Formula I is extracted into an aqueous base solution. It is generally advantageous to first concentrate the liquid portion prior to the extraction. Generally, the liquid portion is concentrated to less than about half its initial volume, preferably to about 20% to about 30% of its initial volume. The concentration can be achieved by a variety of techniques known to those of skill in the art. In one preferred embodiment, the liquid portion is concentrated under vacuum, for example by use of a Rotavap or similar device.

The product (i.e., the compound of Formula I) is then extracted into an aqueous base solution. One convenient technique for the extraction is to add water and an organic solvent to the concentrated solution; adjust the pH of the mixture to a pH of about 11 to about 12; and separate the phases of the pH-adjusted solution. Generally, an amount of water that is from about 100% to about 125% of the volume of the concentrated liquid portion is sufficient, and an amount of organic solvent that is from about 90% to about 110% of the volume of the concentrated liquid portion is sufficient. While a variety of solvents can be used for the extraction, one preferred organic solvent is 1,2-diethoxyethane. The pH is conveniently adjusted by addition of an aqueous solution of a metal hydroxide, for example sodium hydroxide.

Typically, the organic and aqueous phases are then separated, and the organic phase is then extracted with water, and aqueous base, for example 2N sodium hydroxide. The aqueous phases are combined, and optionally washed with an organic solvent, for example 1,2-diethoxyethane. The product can then be collected from the combined aqueous phase by acidifying the aqueous solution, for example by addition of an aqueous solution of a protic acid such as HCl, and recovering the solid product. Preferably, the product is then washed, for example with water.

The product can then be further purified by recrystallization one or more times from a suitable solvent. One suitable solvent is a solution comprising ethanol and water, for example 2:1, v/v. In some embodiments, the recrystallization is performed by suspending the product in alcohol, and heating to a temperature sufficient to dissolve the product, for example about 70° C. to about 80° C. The water is then added while maintaining the elevated temperature.

The purified product is then collected from the solution by cooling, for example to about 0° C. to about 5° C., and physical separation of the solid product from the solution. It is generally advantageous to hold the solution at the cool temperature for a period of time after the cooling is complete, to afford maximal yield of product. Generally, holding the solution at 0° C. to about 5° C., for about an hour or longer, for example up to about 90 minutes, is sufficient.

In some embodiments, it can be advantageous to cool the solution in more than one stage. For example, in some embodiments, the solution is first cooled to an intermediate temperature, for example from about 45° C. to about 50° C., and is then held at that temperature for a period of time, before cooling to lower temperature as described above. Generally, holding the solution at the intermediate temperature for about ten minutes or longer, about twenty minutes or longer, about thirty minutes or longer, or about 45 minutes or longer is sufficient. Preferably, the solution is held at an intermediate temperature of from about 50° C. to about 60° C., more preferably from about 45° C. to about 50° C., for about thirty minutes.

After cooling is complete, the crude purified product can be collected by any convenient means, for example by filtering the solution. Preferably, the product is washed one or more times with a suitable solvent, for example pre-cooled alcohol:water (2:1).

Preferably, the recrystallization as described above is repeated at least once, and the purified product can then be dried by standard procedures, for example at 55° C. to about 65° C., under vacuum, to afford the purified compound.

The processes described herein are useful for the preparation of compounds of Formula I, and especially for the preparation of 2-(3-fluoro-4-hydroxyphenyl)-7-vinyl-1,3-benzoxazol-5-ol.

The processes of the invention typically provide recoveries of compound (relative to the starting material of Formula II) of 40% or greater, 50% or greater, 55% or greater.

The present invention also provides products of the process of the described herein.

As used herein, the term “alkyl” or “alkylene” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like.

As used herein, “alkynyl” refers to an alkyl group having one or more triple carbon-carbon bonds. Example alkynyl groups include ethynyl, propynyl, butynyl, pentynyl, and the like.

As used herein, “halo” or “halogen” refers to fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

The optionally substituted alkyl, alkenyl and alkynyl moieties are each independently optionally substituted by one or more substituents independently selected from the list specified. In particular embodiments the moieties are substituted by 1 to 6 substituents independently selected from the list specified. In further embodiments the moieties are substituted by 1 to 3 substituents independently selected form the list specified.

Trifluoroalkyl and trifluoroalkoxy moieties are preferably 1 to 6 carbon atom straight or branched chain groups. Some suitable embodiments include trifluoroalkyl of 1 to 3 carbon atoms, or trifluoroalkoxy of 1 to 3 carbon atoms each of which can be straight or branched chain e.g. trifluoromethyl and trifluoromethoxy.

When used herein the term aryl refers to a 6-10 carbon atom mono or bicyclic aromatic group e.g. phenyl and naphthyl.

In certain embodiments R₁ is vinyl or 1-propen-2-yl, preferably vinyl. In some embodiments, suitable examples each of R₂, R₂ₐ, R₃ and R₃ₐ may be hydrogen. In certain embodiments R₂ and R₂ₐ are both hydrogen. In certain embodiments R₃ and R₃ₐ are both hydrogen. In some preferred embodiments R₂, R₂ₐ, R₃ and R₃ₐ are all hydrogen. In some embodiments, X is preferably 0. In some embodiments, R₈ is chloride, bromide or iodide, preferably bromide. In some embodiments, the alkene reacted with the compound of formula II is ethene or 1-propene, preferably ethene.

At various places in the present specification substituents of compounds of the invention are disclosed in groups or in ranges. It is specifically intended that the invention include each and every individual subcombination of the members of such groups and ranges. For example, the term “C1-6 alkyl” is specifically intended to individually disclose methyl, ethyl, C3 alkyl, C4 alkyl, C5 alkyl and C6 alkyl.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The processes of this invention are suitable for the purification of compounds of Formula I on any convenient scale, for example greater than about 0.01 mg, 0.10 mg, 1 mg, 10 mg, 100 mg, 1 g, 10 g, 100 g, 1 kg, 10 kg or more. The processes are particularly advantageous for the large scale (e.g., greater than about ten gram) purification.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLE 1

### Preparation of 2-(3-Fluoro-4-hydroxyphenyl)-7-vinylbenzoxazol-5-ol

A 2 gallon hydrogenator was charged with 2-(3-Fluoro-4-hydroxyphenyl)-7-bromobenzoxazol-5-ol (300 g, 0.926 mole), tri-o-tolylphosphine (9.1 g, 3.3%), palladium diacetate (2.1 g 1%), acetonitrile (4.5 L), and triethylamine (375 g, 4 eq). The hydrogenator was flushed with nitrogen, and with ethylene; and then the pressure was adjusted to 50 psi. The reaction mixture was heated to 75° C. and held for 16 hours, at which time HPLC sampling indicated 0.2% of starting material remaining. The mixture was cooled to 35-40° C. and filtered through a 0.2μ cartridge, and washed with 1,2-diethoxyethane (1.2 L). The filtrate was concentrated under vacuum to 1.2 L, and water (1.5 L) and 1,2-diethoxyethane (1.2 L) were added. The pH was adjusted to 11-12 by adding 1.4 L of 2N NaOH at 15-20° C. The phases were separated, and the organic phase was extracted with water (300 ml), and 2 N NaOH (20 mL). The combined aqueous phase was washed with 1,2-diethoxyethane (2×900 mL). The pH was adjusted to 2.5-3.5 by adding 500 mL of 4N HCl at 15-20° C. After holding for 4 hours, the solid was filtered off and washed with water (3×200 mL). The product was then recrystallized twice from an ethanol:water solution as described below.

The wet cake was suspended in ethanol (1055 mL) and heated to 74-80° C. While maintaining at 74-80° C., water (422 mL) was added. The solution was cooled to 45-55° C. and held for 0.5 hour, then cooled to 0-8° C. and held for 1 hour. The solid was filtered off and washed with a precooled solution of ethanol:water (2:1) (2×200 mL). The wet cake was then suspended in ethanol (945 mL) and heated to 74-80° C. While maintaining at 74-80° C., water (472 mL) was added. The solution was cooled to 45-55° C. and held for 0.5 hour, then cooled to 0-8° C. and held for 1 hour. The solid was filtered off and washed with a precooled solution of ethanol:water (2:1) (2×200 mL). The product was dried in a vacuum oven at 55-65° C. and 5-10 mm Hg for 24 hours to afford 146 g of 2-(3-Fluoro-4-hydroxyphenyl)-7-vinylbenzoxazol-5-ol (58% yield).

As those skilled in the art will appreciate, numerous changes and modifications may be made to the preferred embodiments of the invention without departing from the spirit of the invention. It is intended that all such variations fall within the scope of the invention.

It is intended that each of the patents, applications, and printed publications including books mentioned in this patent document be hereby incorporated by reference in their entirety.

What is claimed is:

1. A process for preparing a compound of Formula I: 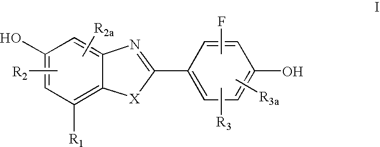
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, —CONR₅R₆, —NR₅R₆ or —N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
comprising:
reacting a compound of Formula II: 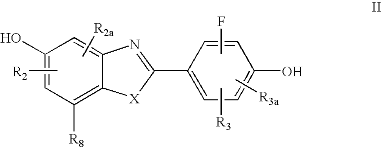
wherein:
R₈ is chloride, bromide, iodide, mesylate, tosylate, triflate, nonaflate or a diazonium salt;
with an alkene having from 2 to about 7 carbon atoms, and which is optionally substituted with hydroxyl, —CN, halogen, trifluoroalkyl, trifluoroalkoxy, —COR₅, —CO₂R₅, —NO₂, —CONR₅R₆, —NR₅R₆ or —NR₅COR₆;
in the presence of a suitable palladium catalyst and a suitable base, for a time and under the conditions effective to form the compound of Formula I.

2. The process of claim 1 wherein the compound of Formula I has the structure: 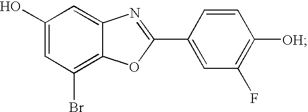
the compound of Formula II has the structure: 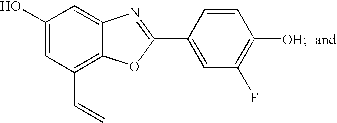
the alkene is ethylene.

3. The process of claim 1 wherein the palladium catalyst comprises a palladium (II) salt and one or more suitable phosphine ligands.

4. The process of claim 1 wherein the palladium catalyst comprises palladium diacetate and tri-o-tolylphosphine.

5. The process of claim 4 wherein the molar ratio of tri-o-tolylphosphine to palladium diacetate is from about 2 to about 4.

6. The process of claim 4 wherein the molar ratio of tri-o-tolylphosphine to palladium diacetate is about 3.3.

7. The process of claim 4 wherein the palladium diacetate is present in an amount of up to about 3 mole percent relative to the compound of Formula II.

8. The process of claim 1 wherein the palladium catalyst comprises palladium diacetate and tri-o-tolylphosphine, the molar ratio of tri-o-tolylphosphine to palladium diacetate is from about 2 to about 4, and the palladium diacetate is present in an amount of up to about 3 mole percent relative to the compound of Formula II.

9. The process of claim 1 wherein the base comprises a nitrogen base.

10. The process of claim 1 wherein the base comprises a trialkyl amine.

11. The process of claim 1 wherein the base comprises triethylamine.

12. The process of claim 1 wherein the molar ratio of the base to the compound of Formula II is from about 4 to about 8.

13. The process of claim 1 wherein the base comprises a trialkyl amine, and the molar ratio of the base to the compound of Formula II is from about 4 to about 8.

14. The process of claim 1 wherein the base comprises triethylamine, and the molar ratio of the base to the compound of Formula II is about 4.

15. The process of claim 1 wherein the reaction is performed in a solvent comprising a polar organic compound.

16. The process of claim 1 wherein the solvent comprises acetonitrile.

17. The process of claim 1 wherein the reaction is performed at a temperature of less than about 100° C.

18. The process of claim 1 wherein the reaction is performed at a temperature of from about 50° C. to about 100° C.

19. The process of claim 1 wherein the reaction is performed at a temperature of from about 70° C. to about 80° C.

20. The process of claim 1 wherein the reaction is performed under a pressure greater than atmospheric pressure.

21. The process of claim 1 wherein the reaction is performed under a pressure of about 50 psi.

22. The process of claim 1 wherein the reaction is performed for a time of up to about 24 hours.

23. The process of claim 1 wherein the reaction is performed for a time of up to about 16 hours.

24. The process of claim 1 wherein:
the palladium catalyst comprises palladium diacetate and tri-o-tolylphosphine;
the molar ratio of tri-o-tolylphosphine to palladium diacetate is from about 2 to about 4;
the palladium diacetate is present in an amount of up to about 3 mole percent relative to the compound of Formula II;
the base comprises a trialkyl amine; and
the molar ratio of the base to the compound of Formula II is from about 4 to about 8.

25. The process of claim 2 wherein:
the palladium catalyst comprises palladium diacetate and tri-o-tolylphosphine;
the molar ratio of tri-o-tolylphosphine to palladium diacetate is from about 2 to about 4;
the palladium diacetate is present in an amount of up to about 3 mole percent relative to the compound of Formula II;
the base comprises a trialkyl amine; and
the molar ratio of the base to the compound of Formula II is from about 4 to about 8.

26. The process of claim 25 wherein the reaction is performed in a solvent comprising a polar organic compound.

27. The process of claim 26 wherein the reaction is performed in a solvent comprising acetonitrile.

28. The process of claim 26 wherein the reaction is performed at a temperature of from about 50° C. to about 100° C.

29. The process of claim 2 wherein:
the palladium catalyst comprises palladium diacetate and tri-o-tolylphosphine;
the molar ratio of tri-o-tolylphosphine to palladium diacetate is about 3 to about 4;
the palladium diacetate is present in an amount of about 1 mole percent relative to the compound of Formula II;
the base comprises triethyl amine;
the molar ratio of the base to the compound of Formula II is about 4;
the reaction is performed at a temperature of from about 50° C. to about 100° C., and at a pressure greater than atmospheric pressure; and
the solvent comprises acetonitrile.

30. The process of claim 29 wherein the reaction is performed at a temperature of from about 70° C. to about 80° C., and at a pressure of about 50 psi.

31. The process of claim 30 wherein the reaction is performed for up to about 16 hours.

32. The process of claim 1 further comprising:
a) separating the liquid portion of the resulting reaction mixture from the solid portion;
b) optionally washing the solid portion with an organic solvent and combining the wash with the separated liquid portion;
c) concentrating the liquid portion;
d) extracting the compound of Formula I into an aqueous base solution;
e) acidifying the aqueous base solution; and
f) collecting the compound of Formula I.

33. The process of claim 32 wherein step (d) comprises:
i) adding water and an organic solvent to the concentrated solution;
ii) adjusting the pH of the mixture resulting from step (i) to a pH of about 11 to about 12;
iii) separating the phases of the pH-adjusted solution, providing an organic phase and an aqueous phase;
iv) extracting the organic phase with aqueous base;
v) combining the aqueous phase and the aqueous extracts from step (iv) to form a combined aqueous phase; and
vi) optionally washing the combined aqueous phase with an organic solvent.

34. The process of claim 33 wherein in step (a), the separating is performed by filtration.

35. The process of claim 33 wherein in step (b), the organic solvent comprises 1,2-diethoxyethane.

36. The process of claim 33 wherein in step (c), the liquid portion is concentrated to about 20% to about 30% of its initial volume.

37. The process of claim 34 wherein in step (i), the organic solvent is 1,2-diethoxyethane.

38. The process of claim 34 wherein in step (i), an amount of water is added that is from about 100% to about 125% of the volume of the concentrated liquid portion.

39. The process of claim 34 wherein in step (i), an amount of organic solvent is added that is from about 90% to about 110% of the volume of the concentrated liquid portion.

40. The process of claim 34 wherein in step (ii), the pH is adjusted by addition of an aqueous solution of a metal hydroxide.

41. The process of claim 34 wherein in step (iv), the aqueous base is an aqueous solution of a metal hydroxide.

42. The process of claim 34 wherein in step (vi), the organic solvent is 1,2-diethoxyethane.

43. The process of claim 33 wherein in step (e), the aqueous base solution is acidified by addition of an aqueous solution of a protic acid.

44. The process of claim 43 wherein the protic acid is HCl.

45. The process of claim 33, further comprising recrystallizing the collected compound of Formula I at least once from a solution comprising ethanol and water.

46. The process of claim 33, further comprising recrystallizing the collected compound of Formula I at least once from a solution comprising ethanol and water (2:1, v/v).

47. A product of the process of claim 1.

48. A product of the process of claim 2.

49. A product of the process of claim 8.

50. A product of the process of claim 30.

51. A product of the process of claim 33.

52. A product of the process of claim 34.
